(19) United States
(12) Patent Application Publication
(10) Pub. No.: US 2007/0119465 A1
(43) Pub. Date: May 31, 2007

(54) Biogeometry shapes & methodology for biological energy balancing

(76) Inventor: Ibrahim Fahmy Karim, (US)

Correspondence Address: BROWDY AND NEIMARK, P.L.L.C.;624 NINTH STREET, NW, SUITE 300, WASHINGTON, DC, 20001-5303, US

(21) Appl. No.: 10/585,139

(22) PCT Filed: Dec. 31, 2003
(86) PCT No.: PCT/EG03/00017

§ 371 Date: Jun. 30, 2006

(51) Int. Cl. A61B 19/00; A61M 21/00; A61B 5/00

(52) U.S. Cl. 128/897; 607/88; 600/300; 600/26

(57) ABSTRACT

Geometric shapes and techniques, along with the methodology behind them, invented to produce a qualitative energy balancing effect on biological systems, be it human beings or otherwise. This results in a positive effect on biological functions, either preventative or healing, and protection from environmental energy disturbances. The methodology introduced by the inventor is the discovery of the qualitative energy components of earth energy power-spots {specifically those components are qualitative higher harmonics of the frequencies of gold and ultra-violet, and a carrier wave in the negative green of the energy spectrum} and development of geometric shapes and techniques to reproduce and amplify that energy balancing quality. This methodology has been termed/copyrighted “BioGeometry” by the inventor. The geometric shapes and techniques work either directly through proximity to the biological system or from within its surrounding environment, or by superimposing on a radiating energy source that reaches the biological system. The two techniques are: (1) Using two of the shape (FIG. 4) as geometric polarizers and orienting them in relation to a central axis to produce the energy balancing quality as in (FIG. 7) and termed “BioGeometry Dial”, (2) Using shape (FIG. 4) as an individual geometric polarizer and aligning samples of materials from the biological system itself or its environment to produce the energy balancing effect (FIG. 8) and termed “Energy Balancing Wheel” when in two-dimensional form and “Energy Balancing Sphere” when in three-dimensional form.

Geometric shapes and techniques, along with the methodology behind them, invented to produce a qualitative energy balancing effect on biological systems, be it human beings or otherwise. This results in a positive effect on biological functions, either preventative or healing, and to protect from environmental energy disturbances. The methodology introduced by the inventor is the discovery of the qualitative energy components of earth energy power-spots {specifically those components are qualitative higher harmonics of the frequencies of gold and ultra-violet, and a carrier wave in the negative green of the energy spectrum} and development of geometric shapes and techniques to reproduce and amplify that energy balancing quality. This methodology has been termed/copyrighted “BioGeometry” by the inventor. The geometric shapes and techniques work either directly through proximity to the biological system or from within its surrounding environment, or by superimposing on a radiating energy source that reaches the biological system. The present invention is not rigid in its scope of application, but is rather a dynamic design language that can be applied in any field.

BioGeometry is a science that deals with the energy of shape; it uses shapes, colours, motion, orientation and sound to produce a vibrational quality that balances energy fields. BioGeometrical shapes are two or three-dimensional shapes specially designed based on the energy principles of shape and the interaction with the earth's energy fields to produce balancing effects on the multiple energy levels of biological systems. The discovery of the qualitative energy components of the earth's power-spots mentioned above, which are the measurable indicators of the existence of a transcendental energy that qualitatively balances the micro-vibrational energy structure of biological systems, is the basis for the efficacy of BioGeometry shapes and techniques, which replicate and amplify that qualitative energy environment within biological systems.

BioGeometry can be better understood by an introduction to the physics of qualitative energy interactions from this paper by Dr. Karim on the BioGeometry Energy Systems Ltd. website titled “The Physics of Quality:”

Nature does not recognize our scientific units of measurement. The centimeter, inch, mile, volt etc., do not exist in nature, they are our way of understanding nature by quantifying it. When nature recognizes quantity, it does so by interacting with the “qualitative aspect of quantity”. In nature for example every number has a vibratory quality that can be transmitted through interaction and can produce certain effects on other energy systems. Quality and quantity are actually two faces of the same coin.

Pythagoras was the first to introduce to the western world the Ancient Egyptian way of correlating musical qualities with quantifiable, numerical values. He used a simple monochord, which is made of a musical chord strung on a piece of wood, and showed that every quality of sound played on the chord was based on a certain length on the string, showing that we have in a simple monochord an instrument that can actually convert quality into quantity and vice versa. Every musical instrument performs this conversion.

To understand the properties of this qualitative scale, we look at the musical notes and their relationship to the quantitative frequency of sound or the length of the musical chord. The fact is that at certain intervals the chords interact. When one cord is struck others also start vibrating, in what we call resonance, these are labeled as the same musical note, although they are at different lengths on the instrument, or levels on the scale, because they produce the same effect on the nerves going from the ear to the b rain, and therefore are perceived by the nervous system as the same quality hence the same note. Any trained musician has usually acquired enough sensitivity to perceive the quality or musical note of any one of the sounds produced anywhere on the strings of an instrument even if they sound quite different to the layman. This repetitive or cyclic effect is the reason why we speak of octaves on the piano because the first, eighth, Fifteenth notes and so on, produce the same qualitative effect on us.

From that perspective we can assume that all senses produce similar patterns of nervous reaction to external stimuli. The problem lies in the ability to measure, and measurement must be based on a scale. If we had measurement scales for quality, then it would become objective, and even quantifiable, which would then become scientific. Do such qualitative scales exist? They actually do exist within our brain. We have musical scales, colour scales, and in a less obvious way, also scent and touch scales.

There are two different laws applying to each type of scale, one specific to that scale within its sensory range, and another of a transcendental nature applicable to all vibratory ranges. It is the latter type that interests us, because it allows us to import some of the qualitative laws of each sense scale into the other. If we could build a set of “universal laws of quality”, we would immediately open new dimensions and see new possibilities. As an example we could apply the laws of resonance, as found in sound, to colour, and would understand that similar colours can enter into resonance thus amplifying their energy effect. As in music we will find that similar movements, shapes, feelings, thoughts etc. could also enter into resonance with each other amplifying the vibratory interaction and information exchange that occurs in resonance.

Even though the same laws apply to all sense scales, we will leave other scales such as, touch, taste, and smell out for the moment, as they are not widely used by instruments of qualitative measurements, and will refer to them when relevant, or when discussing instruments that use them.

In order to understand the difference between the perceived colour and the qualitative effect of colour on our systems, if we for example show a subject a red coloured paper we will get reactions on two levels. The first obvious subjective effect is that he sees a red coloured paper and reacts to it based on his conscious and subconscious associations. On another level which is objective his system reacts to the effect of the quality of vibratory frequency on his energy field as part of the colour effect on him. If we however place the red paper out of his visionary field (behind him) but close to his body (within his energy field), we get the unperceived secondary effect only which is purely objective. This second effect is not only in the colour range, as it is beyond the senses, but in all other sensory and extrasensory vibratory ranges as well. As an example the ‘red effect’ or ‘red quality’ can also be found in the auditory sound range as the unperceived effect behind the note ‘do’ or ‘C’. When using the scale of seven colour qualities we are actually dealing with seven abstract qualities existing throughout the whole vibratory universe from the smallest to the largest frequency.

When a person is deprived of one of the senses he still feels an effect through one of the other senses or in an extra sensory way as an example the colour blind still feel the difference between colours and some very sensitive people can feel colours with their hands when blind folded. We then have here seven abstract extrasensory qualities that manifest themselves in any sense scale and enable us to translate any colour into musical notes, smell fragrance, touch, taste or shape quality!!!. This will become clear if we remember that colours can be perceived as a refraction of angles (of different wave lengths) through a glass prism. Angles being the building blocks of shapes, and so the translation of colour into shape becomes evident. From that we can see that as with colour, sound, smell, touch and taste can be translated into shape (and from there the translation into movement is just another step). In the qualitative worldview everything in the universe is interacting in a huge harmonious symphony through the universal language of quality.

The laws of this new ‘Physics of Quality’ seem very similar to the laws of ‘Quantum Physics’, but whereas quantum physics applies to subatomic levels, the physics of quality is universally applicable to the subtle energy interactions at all levels even the ones within what we perceive as the physical level.

When two energy systems (e.g. Observer and observed) are in resonance, information exchange occurs, and afterwards both do not return to their original state. They keep part of the information, which changes both energy systems and keeps them connected forever. This is very much like what has been observed in quantum physics.

I recall that in 1961 during my first year of study of Architecture at the Federal Institute of Technology (FIT) in Zurich, Switzerland, we had art courses with Professor Hans Ess, a prominent figure in the Swiss and German abstract art scene. We were assigned exercises where we were required to transform musical compositions into visual art. We would listen to Bach and transform his “Fugues” into abstract art by transforming musical relationships into; colour, form, and mathematical ratios of composition. Only in later years did I understand the immense importance of these artistic exercises in the understanding and forming of my new concepts of interchangeability of qualitative scales.

I will use one of those scales, the colour scale, from now on to show the difference between ‘Physical Radiesthesia’ and ‘Mental Radeisthesia’ because of my greater familiarity with this particular scale through my training as an architect. Also, because it was used by the French scientists Leon de Chaumery and Antoine de Bellizal, the pioneers of the physics of detection of qualitative effects on the human energy system, in the 1940-1950s in the calibration of the pendular instruments of ‘Microvibratory Physics’ (as in the title of their book) or ‘Physical Radiesthesia’. Other great works were done at the time by Engineer Louis Turrenne using polarity scales, and by Voillaume using pendular length scales. Later on, in Germany the theories of Lecher in Radio wave ‘double’ antennas were used by Schneider to develop the Lecher antenna, used for the detection of the wavelengths that our body energy fields are interacting with. It can also be calibrated to a colour scale for qualitative measurements.

The Lecher antenna is now the main instrument used for the qualitative detection of energy interaction among European practitioners of the science of physical Radiesthesia to be distinguished from the widely popularized form of mental Radiesthesia (better known in England and USA as Dowsing) where pendulums are used as a tool to interact with subconscious levels through a code that gives meaning to the movement of the pendulum in a form of auto-hypnosis (often referred to as automatism). This method of learning to use a pendulum or the movement of a finger to indicate a reaction from the subconscious is a very valuable method to enter into a dialog with the subconscious or for mapping its levels and possibilities. But it is unfortunately very subjective and prone to autosuggestion. It has been widely used in all ancient cultures and has survived as a form of divination used mainly for water detection. However due to its subjective nature, it has acquired a flippant reputation as has been relegated to the divinatory arts. The popularity oft his method of Radiesthesia however has made it necessary to separate it from the scientific approach which uses the scales of measurement on a level of perception that precedes the subjective meaning level as we shall see.

Chaumery and Bellizal named their science Microvibratory physics to differentiate it from the mental psychic form of Radiesthesia. Others applied the term ‘Physical Radiesthesia’ or ‘the physical method of Radiesthesia’ to the scientific form, and plain ‘Radiesthesia’, ‘Mental Radiesthesia’ or more commonly ‘Dowsing’ to the psychic form. This differentiation was also needed because of the apparent similarity in some instruments. In Physical Radiesthesia the instruments are calibrated for measurements and are not dependent on practitioner's personal psychic abilities, which are prone to autosuggestion.

Absolute and perceived reality

Our senses, are the windows of perception through which, we experience a range of frequencies that our organs are sensitive to. Different species have different sensitivity ranges and perceptive possibilities that best serve their adaptability to the environment and survival skills; they experience a very different reality depending on their sensory ranges. The range of frequencies that we perceive through our sense organs produce nervous impulses that are sent to certain areas in the brain and there they are first transformed through sensory scales into colour, sound, touch, smell, and taste. The data from the sense scales interacts with the content of the meaning level of the brain (our individual data banks of information) to form our perceived reality which is based on the interpretation of a small number of very small ranges from the absolute reality.

The absolute reality is the totality of all vibratory ranges of energy in the universe. Even though we interact with the totality of the ranges of the absolute reality, we are only aware of a minute fraction of it through the limitations set by our senses. There are no colours or sounds in the absolute reality. This might sound like a bold statement, but it can be better Understood using the analogy of a simple experiment: if we let light, which has no colour, pass through a glass prism, the light will be refracted at different angles producing the colours of the spectrum. The components which are bent at different angles according to their wavelength are separated and become visible as different colours, each on its own. If we reverse the process and put all the colours together, that means every colour and its opposite, which are also called complementary will be merged, and the original colourless whole light will be reproduced. Similarly if we put all the vibratory ranges of absolute reality together we get the coulourless, and soundless, everything and nothing, that the physicists call “energy soup”. The absolute reality is all energy, and every species of being interacts with it through different senses and sense ranges to produce its own perceived reality through which it can achieve optimal interaction in its physical environment.

In order to understand how this process works and where the different scales are, we will separate the sense processes into several levels of interaction. The first level at the sense organ, where a specific range of vibration is selected from the total vibratory range of energy that exists within the absolute reality. Each sense organ is designed to select the limited vibratory range with which it interacts.

At the second level the information from the first level of interaction is converted and transferred as electrical data along the nerve leading to a specific area in the brain related to that specific organ. At this second level, the nervous reaction to the information of the different sense organs is the same; either an increase or decrease in the different components of the electrical nervous impulses. No colour, sound, smell, taste, or touch sensation is transferred through the nerve. All nerves are the same they all transfer electrical impulses which can carry certain codes of information as a result of the reaction to the energy received by the sense organ.

The third level is where our nervous reaction is evaluated according to a qualitative scale of colour, sound, smell, taste, or touch. This is where the same type of nervous impulse from the second level is translated into a diversity of perceptions. In the third level, the translation is abstract, objective to all human beings, because they all have a similar anatomy. On that level these scales do not only indicate quantitatively the level of increase or decrease of different aspects of the electrical nervous impulses, but also other non-quantitative aspects of qualitative nature. We actually do not perceive the quantitative aspect of those scales of perception. We perceive in the colour red, the sound note C or Do, the taste sweet, etc. certain qualities and not quantities. That is why we will refer to them as qualitative scales of measurement.

The measure of quantity is linear, and when applied to vibration it would be a measure of the speed of movement, that we call frequency, and measure in Hertz units. The possibilities of our scientific instruments put the limits to what we can measure, at both ends of the scale: the very small or the very big. The present level of technology will therefore always limit our traditional scientific worldview. The third level of sense perception, is an abstract objective qualitative measurement according to the scales of colour,sound, smell, taste and touch. There is no meaning yet on this level, it is just the abstract transformation according to the scales.

The Fourth Level of Perception is the meaning level of perception. In the brain all the different types of information from the different scales of perception are again put together and correlated with the memory contents of the brain, our ever expanding personal data banks, to produce the final form of perceived reality as we know it. This is the meaning level that puts our space-time awareness into the perceived reality. It is actually through the addition of meaning to the results of the scales that the perceived reality is put together.

In the fifth level this perceived reality is projected outside onto its' respective areas of absolute reality. With the selection of the senses the brain produces the world around us with its shapes, colours, sounds, smells and so on. These criteria are projected from inside our brains on the absolute reality.

It seems that through some type of wave interference the picture is recreated holographically in a three dimensional form outside our brain to appear outside of us. The holographic 3D image is within a vibratory range that we perceive as physical.

The absolute reality will have every colour, sound, etc. and its' opposite. Which means, everything put together without selection giving a total energy soup containing everything that if perceived in its' totality would produce nothing. A nothing that includes everything, just like putting all the colours of the spectrum together would give the full light which is colourless. Similarly, all sounds put together would cancel each other out. So the absolute reality is everything and nothing.

This also applies to time and space, which is created by our reality, and differs from other time space configurations of other species, or other dimensions. In our dreams for Example, time seems to run at different speeds and space follows different laws than in our waking state. Time-space concepts of every organism differ according to its environment.

In Physical Radiesthesia we are measuring the quality of interaction at the third level which is before the meaning level which is usually coloured by our different data banks. We apply the colour or polarity scale to all levels of vibration inside or outside the boundaries of our perception to become a universal scale of quality measurement. The universal application of the polarity scale to all vibratory levels is more understandable as everything is energy, and energy is vibration, and vibration is the interplay of positive and negative polarities. But one can easily substitute the colour red for positive polarity and blue or violet for the negative polarity. Or red for increasing and blue for decreasing energy functions. By using those scales in a universal way one can access realms beyond the limits of quantitative science, however in a qualitative way. The qualitative way makes possible the interaction with all existing levels of energy in nature, which include the physical, vitality, emotional, mental, and spiritual levels in an abstract objective way. The linear character of quantitative measurement, which is bound by time and space, gives way to the non-linear character of qualitative measurements that have no time or space boundaries.

In 1973 during my visit to Paris on a government mission for the Egyptian Ministry of Health, to which I was a planning advisor to the Minister, I was lucky enough to obtain all the works on Physical Radiesthesia which were buried in one of the rooms in the cellar of the “Maison de la Radiesthesie” in Rue St. Roche in Paris (which does not exist any more and not to be mixed with the new bookshop with that name near the Madeleine). The owner Madanie Lambert who was over eighty years old at the time, told me that Monsieur Lambert, her husband and founder of the center, who was a famous radiesthesist at the time, knew Chaumery, Bellizal and Turrenne very well. They were in constant exchange of views between the two types of Radiesthesia, but the physical method never really caught on, due to its' lack of efficiency as a psychic method of divination, which had made traditional Radiesthesia very popular at the time. And also because of the tragic accident that caused the death of Chaumery who was a victim of one of his experiments on mummification using geometrical shapes to emit energy, which stopped research in the field for many years. She gave me all the old books and instruments of the scientists of that era.

This started the beginning of a journey that ended in the sorting out of this science based on the modern concepts of physics and Pythagorean and Ancient Egyptian theories. This is what I have developed into a science of qualitative measurement that we today refer to as Egyptian Radiesthesia. This then became the research method that when combined with Pythagorean harmonics and research into perception led to the development of the new qualitative physics and opened the way to research into the realm of Earth's subtle energies at sacred power spots that sprouted the holistic universal science of BioGeometry.

BioGeometry is a design language of colour, form, motion, and sound to produce energy balance in all living and inert systems of the environment. In a physics of quality the scale used whether colour, musical notes, or any other, is just one part of the assessment. An energy system, whether animate or inanimate, is a living entity that has a pattern of arrangement in which the qualities take their place to produce the final unity and harmony of the energy system. Just like a pattern of arrangement is used in placing musical notes in a sequence with specific intervals gives the final composition that we perceive as music. Another example is the geometrical pattern of a molecule into which the atoms are arranged. It is the pattern which can be seen as the geometrical shape of arrangement of the components that gives the final qualities, not found in the components themselves, to the molecule. This spatial arrangement, whether as musical intervals, or geometrical configuration, is what achieves the synergy where the final qualities are much beyond what can be found in the components themselves. This is the pattern that we perceive as the quality of the energy system as a whole.

There is a specific energy pattern of arrangement for every system that gives it its properties. Every energy pattern or grid must itself combine the qualities in a perfect balance for it to function properly. The state of perfect balance of the grid can also be detected through measurement using any of the quality scales. There seems to be a very specific energy quality that can be detected when systems are in perfect balance. This energy quality is linked to a source beyond the time-apace frame of the system itself. The balancing seems to come from a transcendental source. This can be understood when we take the analogy of the geometrical circle where the balance of the shape comes from the center point. Whenever a system is in perfect balance we find the three basic quality components of BioGeometry: A higher harmonic of Gold quality, a higher harmonic of the ultra violet quality, and a geometrical carrier wave referred to as negative green in Physical Radiesthesia.

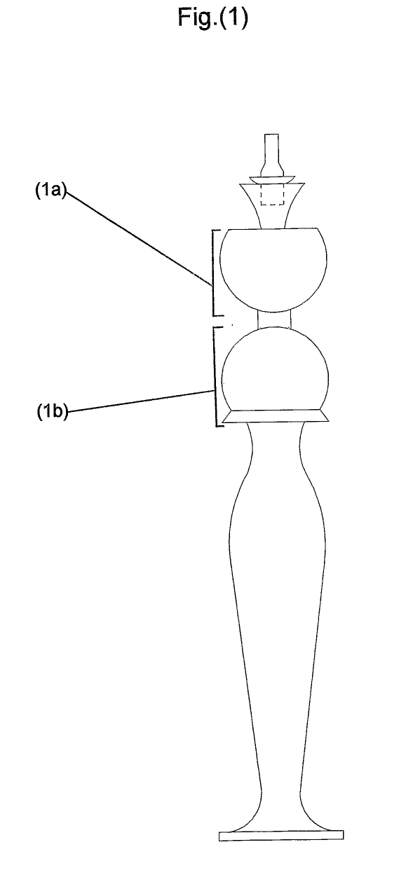

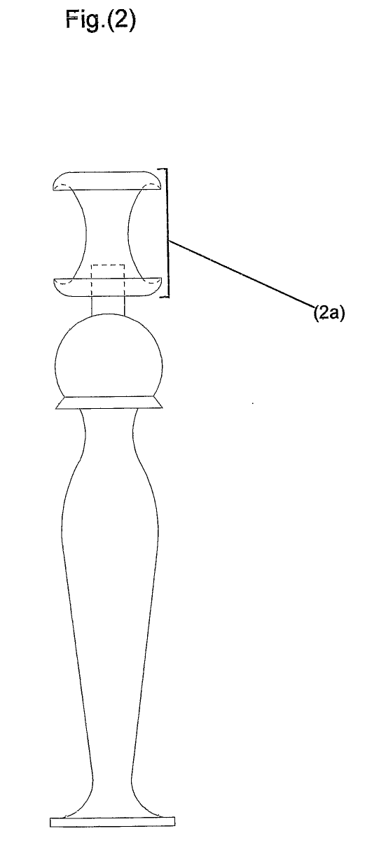

1. Geometric shapes (FIGS. 1,2,3,4,5,6) and techniques producing a qualitative energy balancing effect on biological systems, resulting in a positive effect on biological functions and protection from environmental energy disturbances.

2. A methodology and geometric shapes (FIGS. 1,2,3,4,5,6) according to claim 1, replicating and amplifying the three components {qualitative higher harmonics of gold and ultra-violet, as well as carrier wave in the negative green of the energy spectrum} of earth's energy power-spots.

3. Geometric shapes (FIGS. 1,2,3,4,5,6) according to the preceding claims, in or on any material.

4. Geometric shapes (FIGS. 1,2,3,4,5,6) according to the preceding claims, in any size.

5. Geometric shapes (FIGS. 1,2,3,4,5,6) according to the preceding claims, the same composition in different proportions.

6. Geometric shapes (FIGS. 1,2,3,4,5,6) according to the preceding claims, in either in two or three dimensional form.

7. A geometric shape (FIG. 1) according to the preceding claims, with at least one extended hemisphere (1a) and at least one adjusted hemisphere (1b).

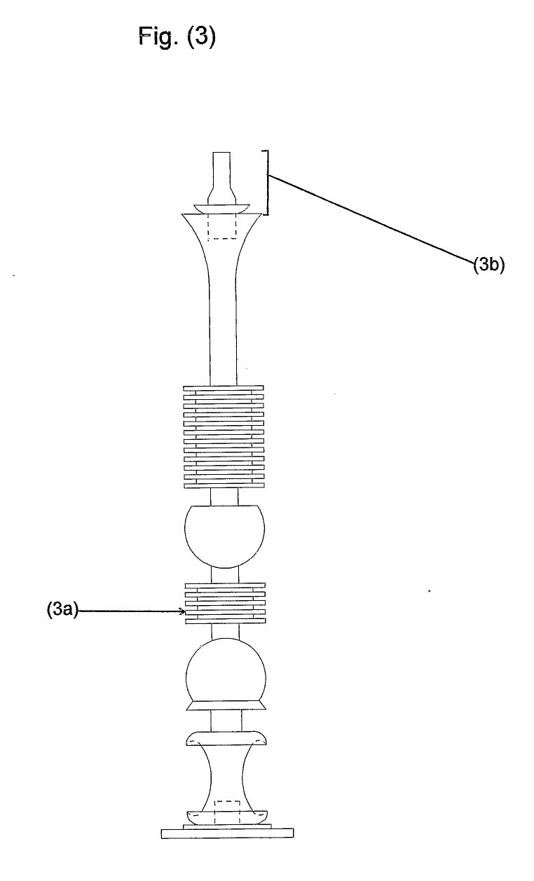

8. A geometric shape (FIG. 2) according to claims 1 through 6, with at least one taurus (2a) and at least one adjusted hemisphere (1a).

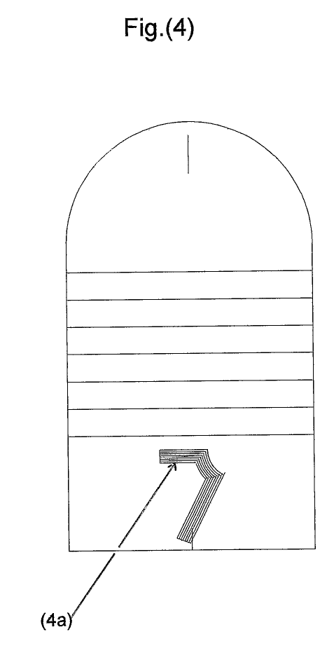

9. A geometric shape (FIG. 3) according to claims 1 through 6, with at least one taurus (2a), at least one extended hemisphere (1a), at least one adjusted hemisphere (1b), at least one disc (3a), and with or without the BioGeometry Dial (3b).

10. Geometric shapes (FIGS. 1,2,3) according to claims 1 through 6, with or without insertions (shown in dotted/broken lines in the drawings).

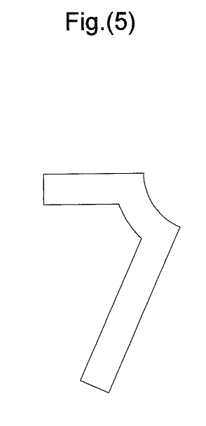

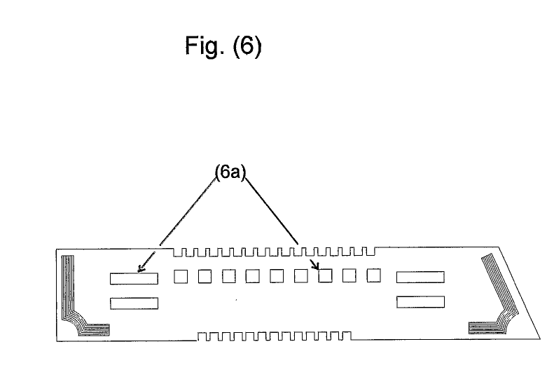

11. A geometric shape (FIG. 4) according to claims 1 through 6, with at least one linear L-design (4a).

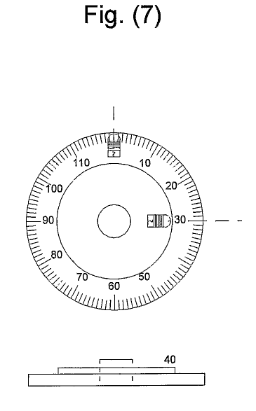

12. A geometric shape (FIG. 5) according to claims 1 through 6, in any thickness.

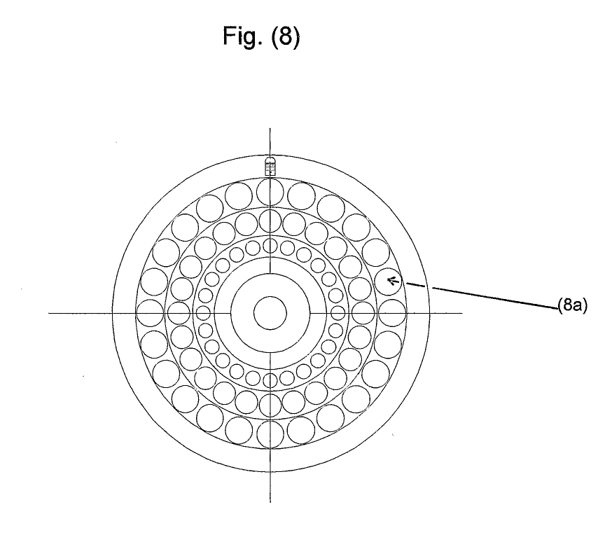

13. A geometric shape (FIG. 6) according to claims 1 through 6, with at least one opening (6a), and at least one linear L-design (4a)

14. A methodology according to claims 1 and 2, of using at least two of shape (FIG. 4) as geometric polarizers and orienting them along a central axis at the required orientation for energy balancing.

15. A device “BioGeometry Dial” according to claim 13, with at least two rotating dials on a central axis with the geometric polarizers FIG. 4).

16. A methodology according to claims 1 and 2, of using at least one shape (FIG. 4) as a geometric polarizer, and orienting material samples from the biological system or its environment to achieve energy balancing.

17. A device according to claim 15, with at least one geometric polarizer and at least one opening to place the samples (8a). Termed “BioGeometry Energy Balancing Wheel” when in two-dimensional form and “BioGeometry Energy Balancing Sphere” when in three-dimensional form.

18. Geometric shapes according to the preceding claims, termed “BioGeometry.”
